(19) United States
(12) Patent Application Publication
(10) Pub. No.: US 2022/0395984 A1
(43) Pub. Date: Dec. 15, 2022

(54) Nursing robot for monitoring critical patients

(71) Applicant: NANJING GONGCHENG QIANGQIANG SOFTWARE TECHNOLOGY CO.LTD, Nanjing, Jiangsu (CN)

(72) Inventor: Yefei ZHANG, Nanjing, Jiangsu (CN)

(21) Appl. No.: 17/286,460

(22) PCT Filed: Nov. 5, 2020
(86) PCT No.: PCT/CN2020/126612

§ 371 Date: Apr. 17, 2021

(30) Foreign Application Priority Data
Oct. 13, 2020 (CN) 202011088292.0

(51) Int. Cl. B25J 11/00; B25J 19/02; A61B 5/00; G06V 40/16; G06V 40/20

(52) U.S. Cl. CPC B25J 11/009; B25J 19/023; A61B 5/0013; A61B 5/746; G06V 40/174; G06V 40/20

(57) ABSTRACT

The invention provides a nursing robot for monitoring critically ill patients, which relates to the field of medical robots, including a robot body, an image taking module arranged at the head of the robot body, a walking device arranged at the bottom of the robot body, a power source module, a processor module, a path finding module, a wireless communication module and a vital sign monitor sampling device arranged in the robot body The video acquisition module is used to collect the video data of the target and send the video data to the cloud server through the wireless communication module. The vital signs monitor sampling module is used to collect the monitoring data of the vital signs monitor and analyze the monitoring data through the processor module to predict the target The development of the disease.

### CROSS REFERENCE TO RELATED APPLICATION

The present application is filed based on and claims priorities from the Chinese Patent Application 202011088292.0 filed Oct. 13, 2020 and PCT/CN2020/126612 filed on Nov. 5, 2020, the content of which is incorporated herein in the entirety by reference.

### TECHNICAL FIELD

The invention relates to the field of medical robot, in particular to a nursing robot for monitoring critical patients.

### BACKGROUND TECHNOLOGY

With the development of artificial intelligence technology, the medical industry has also undergone great changes. Medical robots are gradually participating in some auxiliary work to help medical personnel create higher efficiency. The existing medical robots (medical robots) mainly include: operation robot, rehabilitation robot, remote presentation robot, pharmacy automation robot and nursing robot. In recent years, neural network algorithm and processor chip supporting neural network operation have been constantly innovating, and computer vision system is becoming more and more intelligent. Using computer vision system can not only complete traditional video monitoring tasks, but also further identify the behavior state of target tasks in video through image analysis. However, the existing machine vision system of nursing robot can recognize the behavior state of the target tasks in video It is still at the level of video shooting only by using cameras, so that the machine vision system of nursing robot plays an important role in the same way as traditional surveillance cameras. When monitoring some critically ill patients, we can not find out the patients' differences in time and delay the treatment time. Therefore, how to use the nursing robot to effectively monitor the critical patients becomes a need to be solved Technical issues to be resolved.

### SUMMARY

The invention aims at solving at least one of the technical problems existing in the prior art or related technology, and provides a nursing robot for monitoring critical patients, which can monitor the state and dynamics of patients in real time, discover the abnormal condition in time, and avoid delaying the treatment opportunity under the condition of reducing the input of human monitoring.

The invention is realized through the following technical solutions: a nursing robot for monitoring critical patients, including: a robot body, an image taking module arranged at the head of the robot body, a walking device arranged at the bottom of the robot body, a power supply module, a processor module, a path finding module, a wireless communication module and a vital sign monitor arranged in the robot body The sampling module is used to collect the video data of the target and send the video data to the cloud server through the wireless communication module. The sampling module of the vital signs monitor is used to collect the monitoring data of the vital signs monitor and analyze the monitoring data through the processor module to predict the target For the development of the disease, the image acquisition module, the path finding module, the wireless communication module, the sampling module of the vital signs monitor and the display are all connected with the processor module, the walking device is connected with the path finding module, the alarm device is connected with the wireless communication module, and the power module is the image acquisition module, the path finding module, the wireless communication module, the sampling module of the vital signs monitor, the display, the walking device and the alarm device The alarm device and the processor module are powered.

According to the above technical scheme of the invention, it preferably also includes: an infrared temperature measurement module connected to the power supply module and the wireless communication module, the sensor of the infrared temperature measurement module is set on one side of the image taking module, and the infrared module sends the collected body temperature data to the cloud server through the wireless communication module.

According to the above technical scheme of the invention, preferably, the image taking module specifically includes: laser ranging unit, main camera, depth image acquisition camera and rotating pan tilt, and the driving system of the rotating pan tilt is connected to the processor module.

According to the above technical scheme of the invention, preferably, the sampling module of the vital signs monitor establishes data connection with the vital signs monitor through the connection contact.

According to the above technical scheme of the invention, preferably, the sampling module of the vital signs monitor transmits the collected vital signs data to the cloud server, samples the vital signs data, generates the sampling data change trend diagram, analyzes the sampling data change data diagram according to the preset standard trend diagram, and analyzes the matching degree between the data change trend diagram and the preset image If it is lower than the set threshold, the alarm signal will be sent to the external terminal through the wireless communication module.

According to the above technical scheme of the invention, it is preferred that the vital signs data at least include: blood pressure data, pulse blood oxygen data, body temperature data and respiratory rate data.

According to the above technical scheme of the invention, preferably, the image acquisition module obtains the video image of the target person from multiple angles, and sends the video data to the processor module, which recognizes the dynamic trajectory features of the target person according to the video image, or analyzes the body movement features and facial expression features of the target person according to the video image, and according to the dynamic trajectory features and limb movement features Body movement features or facial expression features can infer the target person's condition.

The beneficial effects obtained by the invention at least include: at the same time of realizing video monitoring, observing critical patients (target persons) through machine vision, data analysis and other means, which is more objective and accurate than manual visit, and is convenient for discovering slight abnormalities of target persons in time, so as to improve the monitoring effect. The range of motion of the robot is improved through the path finding module and the walking device, so as to obtain a better monitoring field of vision. The image acquisition module is used to obtain the image information of the target person, so that the processor module can analyze the image. Based on the image analysis, the abnormal state of the target person can be obtained. Through the wireless communication module, the abnormal state can be sent to the external terminal, so that the medical staff can find the abnormality in time and carry out diagnosis and treatment. The vital signs monitor acquisition module uses the real-time data of the vital signs monitor Once the change trend is different from the set standard trend, it indicates that the patient's condition fluctuates. Timely informing the medical staff of this fluctuation can effectively ensure the safety of critically ill patients.

### ILLUSTRATION

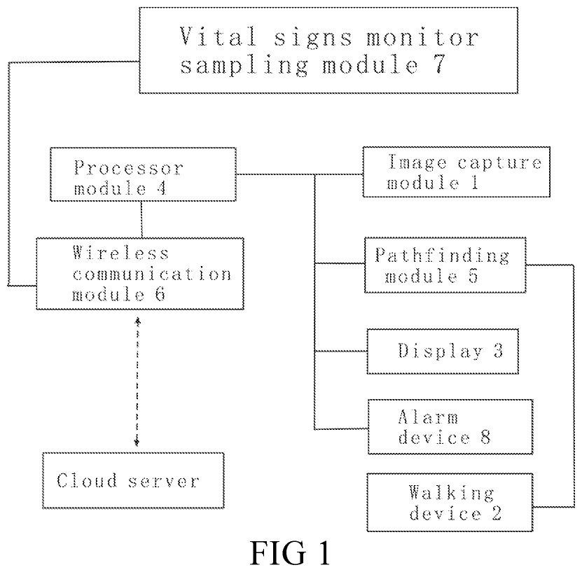

FIG. 1 shows a system structure diagram of a nursing robot for monitoring critical patients according to an embodiment of the present invention.

### SPECIFIC IMPLEMENTATION MODE

In order to better understand the above objects, features and advantages of the invention, the invention will be further described in detail in combination with the drawings and specific embodiments.

As shown in FIG. 1, a nursing robot for monitoring critical patients provided by the invention includes a robot body, an image taking module 1 arranged at the head of the robot body, a walking device 2 arranged at the bottom of the robot body, a power supply module, a processor module 4, a path finding module 5, a wireless communication module 6 and a vital sign monitor sampling module 7 arranged in the robot body, The video acquisition module is used to collect the video data of the target and send the video data to the cloud server through the wireless communication module. The vital signs monitor sampling module is used to collect the monitoring data of the vital signs monitor and analyze the monitoring data through the processor module to predict the disease of the target The image acquisition module, the path finding module, the wireless communication module, the vital signs monitor sampling module and the display are all connected with the processor module, the walking device is connected with the path finding module, the alarm device is connected with the wireless communication module, and the power module is the image acquisition module, the path finding module, the wireless communication module, the vital signs monitor sampling module, the display, the walking device and the alarm device And the processor module.

The infrared temperature measurement module is connected with the wireless temperature measurement module, and the infrared temperature measurement module is connected with the wireless temperature measurement module of the cloud sensor.

Preferably, the image acquisition module specifically includes a laser ranging unit, a main camera, a depth image acquisition camera and a rotating pan tilt, and the driving system of the rotating pan tilt is connected to the processor module.

Preferably, the sampling module of the vital signs monitor establishes a data connection with the vital signs monitor through a connection contact.

Preferably, the sampling module of the vital signs monitor transmits the collected vital signs data to the cloud server, and samples the vital signs data to generate the sampling data change trend diagram. According to the preset standard trend diagram, the sampling data change data diagram is analyzed. If the matching degree between the data change trend diagram and the preset image is lower than the set threshold, the wireless communication is used The module sends an alarm signal to the external terminal. Vital signs data at least include: blood pressure data, pulse oxygen data, body temperature data and respiratory rate data.

Preferably, the image acquisition module obtains the video image of the target person from multiple angles and sends the video data to the processor module, which recognizes the dynamic trajectory features of the target person according to the video image, or analyzes the body action features and facial expression features of the target person according to the video image, and deduces the target person according to the dynamic trajectory features, body action features or facial expression features The condition of the target person was measured.

To sum up, the nursing robot provided by the invention for monitoring critically ill patients not only realizes video monitoring, but also observes critically ill patients (target persons) through machine vision, data analysis and other means, which is more objective and accurate than manual visit, and is convenient for timely discovering slight abnormalities of target persons, so as to improve the monitoring effect. The range of motion of the robot is improved through the path finding module and the walking device, so as to obtain a better monitoring field of vision. The image acquisition module is used to obtain the image information of the target person, so that the processor module can analyze the image. Based on the image analysis, the abnormal state of the target person can be obtained. Through the wireless communication module, the abnormal state can be sent to the external terminal, so that the medical staff can find the abnormality in time and carry out diagnosis and treatment. The vital signs monitor acquisition module uses the real-time data of the vital signs monitor Once the change trend is different from the set standard trend, it indicates that the patient's condition fluctuates. Timely informing the medical staff of this fluctuation can effectively ensure the safety of critically ill patients.

The above description is only the preferred embodiment of the invention and is not used to limit the invention. For those skilled in the art, the invention can have various changes. Any modification, equivalent replacement, improvement, etc. made within the spirit and principle of the invention shall be included in the protection scope of the invention.

1. A nursing robot for monitoring critical patients, which is characterized in that it includes: a robot body, an image taking module arranged at the head of the robot body, a walking device arranged at the bottom of the robot body, a power supply module, a processor module, a path finding module, a wireless communication module and a vital sign monitor sampling module arranged in the robot body, and a setting device An alarm device and a display are arranged outside the body of the robot, wherein the image acquisition module is used to collect the video data of the target and send the video data to the cloud server through the wireless communication module, the vital signs monitor sampling module is used to collect the monitoring data of the vital signs monitor, and the monitoring data is divided by the processor module The image acquisition module, the path finding module, the wireless communication module, the vital signs monitor sampling module and the display are all connected with the processor module, the walking device is connected with the path finding module, the alarm device is connected with the wireless communication module, and the power module is the image acquisition module and the path finding module Power supply is provided by the module, the wireless communication module, the sampling module of the vital signs monitor, the display, the walking device, the alarm device and the processor module.

2. The Nursing Robot for monitoring critical patients according to claim 1, which is characterized in that it also includes: an infrared temperature measurement module connected to the power module and the wireless communication module, the sensor of the infrared temperature measurement module is set on one side of the image taking module, and the infrared module sends the collected temperature data to the cloud service through the wireless communication module Server.

3. The Nursing Robot for monitoring critical patients according to claim 1, which is characterized in that the image acquisition module specifically includes: laser ranging unit, main camera, depth image acquisition camera and rotating pan tilt, and the driving system of the rotating pan tilt is connected to the processor module.

4. The Nursing Robot for monitoring critical patients according to claim 1, which is characterized in that the vital signs monitor sampling module establishes data connection with the vital signs monitor through the connection contact.

5. The Nursing Robot for monitoring critical patients according to any one of claim 1, which is characterized in that the sampling module of the vital signs monitor transmits the collected vital signs data to the cloud server, samples the vital signs data, generates the sampling data change trend diagram, and processes the sampling data change data according to the preset standard trend diagram If the matching degree between the data change trend diagram and the preset image is lower than the set threshold, the wireless communication module will send an alarm signal to the external terminal.

6. The Nursing Robot for monitoring critical patients according to claim 5, which is characterized in that the vital signs data at least includes: blood pressure data, pulse blood oxygen data, body temperature data and respiratory rate data.

7. The Nursing Robot for monitoring critical patients according to any one of claim 1, which is characterized in that the image acquisition module obtains the video image of the target person from multiple angles, and sends the video data to the processor module, and the processor module recognizes the dynamic trajectory characteristics of the target person according to the video image, or according to the video image Image analysis of the body movement features and facial expression features of the target person, and inferring the disease condition of the target person according to the dynamic trajectory features, the body movement features or the facial expression features.

8. The Nursing Robot for monitoring critical patients according to any one of claim 2, which is characterized in that the sampling module of the vital signs monitor transmits the collected vital signs data to the cloud server, samples the vital signs data, generates the sampling data change trend diagram, and processes the sampling data change data according to the preset standard trend diagram If the matching degree between the data change trend diagram and the preset image is lower than the set threshold, the wireless communication module will send an alarm signal to the external terminal.

9. The Nursing Robot for monitoring critical patients according to any one of claim 3, which is characterized in that the sampling module of the vital signs monitor transmits the collected vital signs data to the cloud server, samples the vital signs data, generates the sampling data change trend diagram, and processes the sampling data change data according to the preset standard trend diagram If the matching degree between the data change trend diagram and the preset image is lower than the set threshold, the wireless communication module will send an alarm signal to the external terminal.

10. The Nursing Robot for monitoring critical patients according to any one of claim 4, which is characterized in that the sampling module of the vital signs monitor transmits the collected vital signs data to the cloud server, samples the vital signs data, generates the sampling data change trend diagram, and processes the sampling data change data according to the preset standard trend diagram If the matching degree between the data change trend diagram and the preset image is lower than the set threshold, the wireless communication module will send an alarm signal to the external terminal.

11. The Nursing Robot for monitoring critical patients according to any one of claim 2, which is characterized in that the image acquisition module obtains the video image of the target person from multiple angles, and sends the video data to the processor module, and the processor module recognizes the dynamic trajectory characteristics of the target person according to the video image, or according to the video image Image analysis of the body movement features and facial expression features of the target person, and inferring the disease condition of the target person according to the dynamic trajectory features, the body movement features or the facial expression features.

12. The Nursing Robot for monitoring critical patients according to any one of claim 3, which is characterized in that the image acquisition module obtains the video image of the target person from multiple angles, and sends the video data to the processor module, and the processor module recognizes the dynamic trajectory characteristics of the target person according to the video image, or according to the video image Image analysis of the body movement features and facial expression features of the target person, and inferring the disease condition of the target person according to the dynamic trajectory features, the body movement features or the facial expression features.

13. The Nursing Robot for monitoring critical patients according to any one of claim 4, which is characterized in that the image acquisition module obtains the video image of the target person from multiple angles, and sends the video data to the processor module, and the processor module recognizes the dynamic trajectory characteristics of the target person according to the video image, or according to the video image Image analysis of the body movement features and facial expression features of the target person, and inferring the disease condition of the target person according to the dynamic trajectory features, the body movement features or the facial expression features.
